(19) United States
(12) Patent Application Publication
(10) Pub. No.: US 2023/0293302 A1
(43) Pub. Date: Sep. 21, 2023

(54) ELECTRONIC IMPLANTABLE PENILE PROSTHESIS WITH REMOTE ACTIVATION

(71) Applicant: Boston Scientific Scimed, Inc., Maple Grove, MN (US)

(72) Inventors: Noel Smith, Windgap (IE); Daragh Nolan, Via Youghal (IE); Eduardo Marcos Larangeira, Cork City (IE); Brian P. Watschke, Minneapolis, MN (US); Thomas Sinnott, Enniscorthy (IE)

(21) Appl. No.: 18/183,685

(22) Filed: Mar. 14, 2023

Related U.S. Application Data
(60) Provisional application No. 63/269,439, filed on Mar. 16, 2022.

(51) Int. Cl. A61F 2/26; A61F 2/48

(52) U.S. Cl. CPC A61F 2/26; A61F 2/482; A61F 2250/0001; A61F 2250/0013

(57) ABSTRACT

According to an aspect, an inflatable penile prosthesis includes a fluid reservoir configured to hold fluid, an inflatable member, and an electronic pump assembly configured to transfer the fluid between the fluid reservoir and the inflatable member. The electronic pump assembly includes a pump, an active valve, and a controller configured to receive an external signal to activate an inflation cycle and control at least one of the pump or the active valve to transfer the fluid to the inflatable member. The controller includes a printed circuit board assembly. The printed circuit board assembly includes a microprocessor or a state machine.

### CROSS-REFERENCE TO RELATED APPLICATION

This application claims priority to U.S. Provisional Patent Application No. 63/269,439, filed on Mar. 16, 2022, entitled “ELECTRONIC IMPLANTABLE PENILE PROSTHESIS WITH REMOTE ACTIVATION”, the disclosure of which is incorporated by reference herein in its entirety.

### TECHNICAL FIELD

This disclosure relates generally to bodily implants and more specifically to bodily implants, such as an electronic implantable penile prosthesis with remote activation and/or power charging.

### BACKGROUND

One treatment for male erectile dysfunction is the implantation of a penile prosthesis that erects the penis. Some existing penile prosthesis include inflatable cylinders or members that can be inflated or deflated using a pump mechanism. The pump mechanism includes a pump, implantable in the scrotum, that can be manually squeezed by the user to move fluid from a reservoir into the cylinders, creating an erection. For some patients, the manual pumping procedure may be relatively challenging.

### SUMMARY

According to an aspect, an inflatable penile prosthesis includes a fluid reservoir configured to hold fluid, an inflatable member, and an electronic pump assembly configured to transfer the fluid between the fluid reservoir and the inflatable member. The electronic pump assembly includes a pump, an active valve, and a controller configured to receive an external signal to activate an inflation cycle and control at least one of the pump or the active valve to transfer the fluid to the inflatable member. The controller includes a printed circuit board assembly. The printed circuit board assembly includes a microprocessor or a state machine.

According to some aspects, the inflatable penile prosthesis may include one or more of the following features (or any combination thereof). The electronic pump assembly includes an antenna. The antenna is configured to receive the external signal. The external signal may include a Bluetooth signal. The external signal may include a near-field communication (NFC) signal or another radio frequency (RF) signal. The printed circuit board assembly may include a power source. The power source is configured to be charged by wireless transmissions (e.g., the NFC signal, capacitive or inductive signal, RF signal, etc.). The printed circuit board assembly includes a magnetic sensing circuit, where the controller is configured to detect activation of the inflation cycle in response to the magnetic sensing circuit detecting a presence of a magnetic field. The electronic pump assembly includes an antenna, where the antenna is configured to receive the external signal, and the external signal includes a radio frequency (RF) signal. The electronic pump assembly includes an inductive antenna, where the inductive antenna is configured to receive the external signal, and the external signal includes an inductive signal. The external signal may include an ultrasonic signal. The electronic pump assembly includes a pressure sensor, where the controller is configured to receive a pressure reading from the pressure sensor that indicates a pressure of the inflatable member, and the controller configured to determine that the inflation cycle is activated in response to pressure exceeding a threshold level. The electronic pump assembly includes a first conductive plate, where the controller is configured to determine activation of the inflation cycle in response to a second conductive plate of an external device being placed within a threshold distance of the first conductive plate. The electronic pump assembly includes an acoustic sensor configured to receive an acoustic signal, where the controller is configured to determine activation of the inflation cycle in response to the acoustic signal.

According to an aspect, an inflatable penile prosthesis includes a fluid reservoir configured to hold fluid, an inflatable member, and an electronic pump assembly configured to transfer the fluid between the fluid reservoir and the inflatable member. The electronic pump assembly includes a pump, an active valve, an antenna configured to receive an external signal, a controller configured to determine to activate an inflation cycle based on the external signal and control at least one of the pump or the active valve to transfer the fluid to the inflatable member, and a power source configured to power the controller, where the power source is configured to be recharged by wireless transmissions received via the antenna.

According to some aspects, the inflatable penile prosthesis may include one or more of the following features (or any combination thereof). The wireless transmissions include near-field communication (NFC) transmissions. The wireless transmissions include radio frequency (RF) transmissions. The wireless transmissions include capacitive or inductive transmissions. The controller includes a printed circuit board assembly, and the printed circuit board assembly includes a microprocessor configured to interpret the external signal. The printed circuit board assembly includes a state machine configured to interpret the external signal. The state machine includes a field-programmable gate array or an application-specific integrated circuit.

According to an aspect, a method of controlling an inflatable penile prosthesis includes detecting, by a controller of an electronic pump assembly, an external signal, the controller including a printed circuit board assembly, the printed circuit board assembly including a microprocessor or a state machine, determining to activate an inflation cycle of the inflatable penile prosthesis based on the external signal, and actuating at least one of a pump or an active valve to transfer fluid from a fluid reservoir to an inflatable member. In some examples, the method includes charging a battery of the electronic pump assembly based on wireless transmissions received via an antenna of the electronic pump assembly.

### BRIEF DESCRIPTION OF THE DRAWINGS

FIG. 1A illustrates an inflatable penile prosthesis having an electronic pump assembly according to an aspect.

FIG. 1B illustrates a controller of the electronic pump assembly according to an aspect.

FIG. 1C illustrates an example of a microcontroller of the electronic pump assembly according to an aspect.

FIG. 1D illustrates an example of a state machine of the electronic pump assembly according to an aspect.

FIG. 1E illustrates a plurality of states of the inflatable penile prosthesis according to an aspect.

FIG. 1F illustrates various types of power systems that can be used in the electronic pump assembly according to an aspect.

FIG. 2 illustrates an inflatable penile prosthesis with an electronic pump assembly configured to communicate with an external device using a Bluetooth wireless connection according to an aspect.

FIG. 3 illustrates an inflatable penile prosthesis with an electronic pump assembly configured to communicate with an external device using a near field communication (NFC) wireless connection according to an aspect.

FIG. 4 illustrates an inflatable penile prosthesis with an electronic pump assembly configured to communicate with an external device using a magnetic field according to an aspect.

FIG. 5 illustrates an inflatable penile prosthesis with an electronic pump assembly configured to communicate with an external device using high frequency signals according to an aspect.

FIG. 6 illustrates an inflatable penile prosthesis with an electronic pump assembly configured to communicate with an external device using low frequency inductive signals according to an aspect.

FIG. 7 illustrates an inflatable penile prosthesis with an electronic pump assembly configured to communicate with an external device using ultrasonic signals according to an aspect.

FIG. 8 illustrates an inflatable penile prosthesis with an electronic pump assembly configured to activate an inflation or deflation cycle based on a gripping signal sensed by a pressure sensor according to an aspect.

FIG. 9 illustrates an inflatable penile prosthesis with an electronic pump assembly configured to communicate with an external device using a capacitive signal according to an aspect.

FIG. 10 illustrates an inflatable penile prosthesis with an electronic pump assembly configured to communicate with an external device using an acoustic signal according to an aspect.

FIG. 11 illustrates an inflatable penile prosthesis having an electronic pump assembly according to another aspect.

FIG. 12 illustrates a flow chart depicting example operations of an electronic pump assembly according to an aspect.

### DETAILED DESCRIPTION

This disclosure relates to an inflatable penile prosthesis that includes an electronic pump assembly to transfer fluid between a fluid reservoir and an inflatable member. The electronic pump assembly includes one or more pumps (e.g., electromagnetic or Piezoelectric pumps), one or more active valves, a pressure sensor, and a controller. The controller may control the pump(s) and/or the active valve(s) to inflate or deflate the inflatable member. In some examples, the controller may cause the active valve to be in a closed position and actuate the pump(s) to pump fluid from the fluid reservoir to the inflatable member during the inflation cycle. Upon detection of a target pressure by the pressure sensor (or the expiration of a timer), the controller may deactivate the pump(s). In some examples, when the deflation cycle is activated, the controller may cause the active valve to be in an open position (thereby allowing fluid to flow through the active valve), where the fluid is transferred back to the fluid reservoir.

In some examples, the controller includes a microcontroller and/or microprocessor. In some examples, the controller includes a state machine implemented by application specific integrated circuit(s) and/or field-programmable gate array. In some examples, the state machine includes a plurality of states such as a standby state in which the inflation member is in a deflated state, an inflation state in the inflatable member is inflated to a target pressure, a hold state in which pressure is held at the target pressure, and a deflation state in which the inflatable member is deflated.

In some examples, the controller may wirelessly communicate with an external device (e.g., a computer, a smartphone, tablet, pendant, key fob, etc.) to control the inflatable penile prosthesis (e.g., inflate or deflate the inflatable member). In some examples, the external device is a near field communication (NFC) device (e.g., a smartphone or custom designed controller), where the controller may receive an NFC signal via an antenna on the electronic pump assembly from the external device to inflate or deflate the inflatable member. In some examples, the external device is a Bluetooth device (e.g., a smartphone or custom designed controller), where the controller may receive a Bluetooth signal via an antenna on the electronic pump assembly from the external device to inflate or deflate the inflatable member. In some examples, the external device is a magnet device, where the controller includes a magnet sensing circuit, which actuates in the presence of a magnetic field from the magnet device.

In some examples, the external device is a radio frequency (RF) device, where the controller may receive a radio frequency (RF) signal (e.g., high frequency signals) (e.g., kHz to GHz) via an antenna on the electronic pump assembly from the external device to inflate or deflate the inflatable member. In some examples, the external device is an inductive device, where the controller may receive a low frequency inductive signal via an inductive antenna on the electronic pump assembly from the external device to inflate or deflate the inflatable member. In some examples, the external device is an ultrasonic device, where the controller may receive an ultrasonic signal via an antenna on the electronic pump assembly from the external device to inflate or deflate the inflatable member.

In some examples, the inflation and/or deflation cycle may be controlled without using an external device. In some examples, the inflation cycle is instituted based on an external signal, where the external signal is a grasp action on the penis. For example, a grasp action on the penis may cause compression on the cylinders in order to trigger an inflation cycle (or inflation mode). For example, the pressure sensor may detect an increased pressure level in the inflatable member, where the controller may determine that the increased pressure level corresponds to an inflation activation signal. Deactivation may be detected by a coded series of grasp actions, triggered by an external device, or a timer. In some examples, the controller includes an acoustic sensor configured to sense an acoustic signal. For example, tapping on the abdomen can be detected by the acoustic sensor to trigger an inflation cycle. In some examples, deactivation may be detected by a coded series of taps. In some examples, the external device is a capacitive device (e.g., having a first plate of a capacitor), where the controller includes a second plate of the capacitor. When the first plate is placed in close proximity (e.g., within a threshold distance) to the second plate (e.g., implanted in the patient), a capacitive signal may be received by the controller to cause the inflatable member to be inflated or deflated.

The electronic pump assembly may include a power source such as a primary cell (e.g., no energy transfer is required), a rechargeable battery, energy storage (e.g., capacitor(s)), and/or direct powering without using a storage device. In some examples, low frequency techniques (e.g., inductive power) may enable activation as well as charge or directly power the implantable device. The external charger may be a portable device or a static device such as a flat encapsulated coil (e.g., on a seat of a chair or on a mattress). In some examples, NFC transmissions may enable activation as well as charge or directly power the implantable device. In some examples, VHF and UHF transmissions may enable activations as well as charge or directly power the implantable device. In some examples, capacitive coupling technology may enable activation as well as charge or directly power the implantable device.

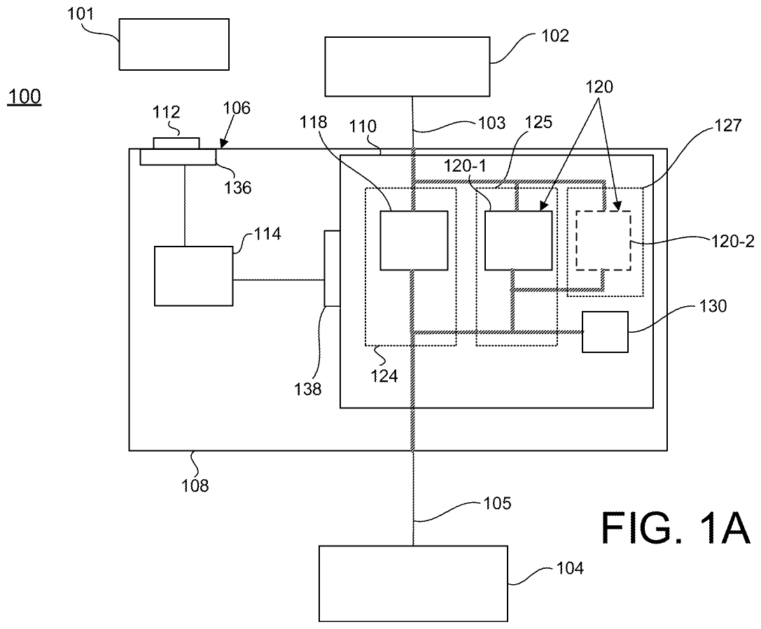

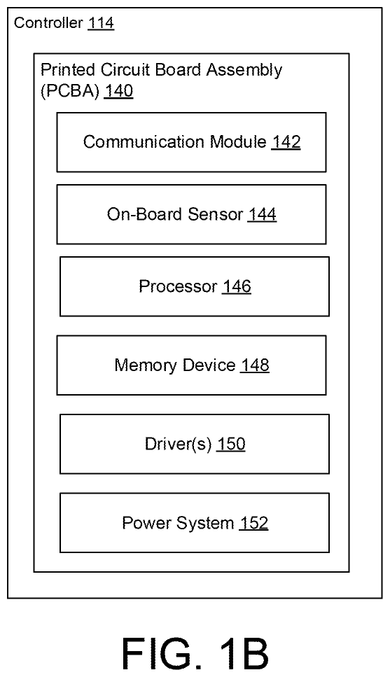

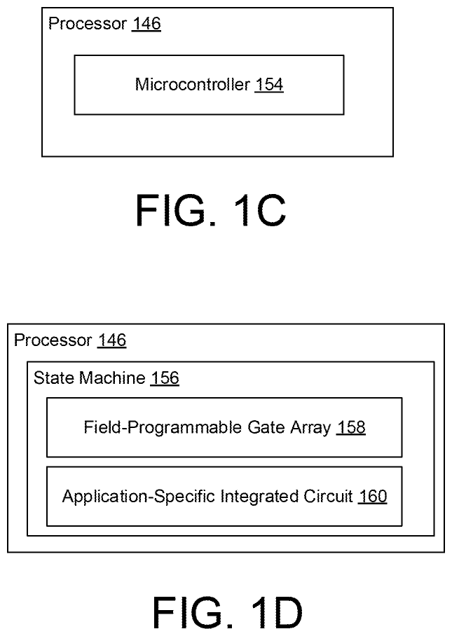

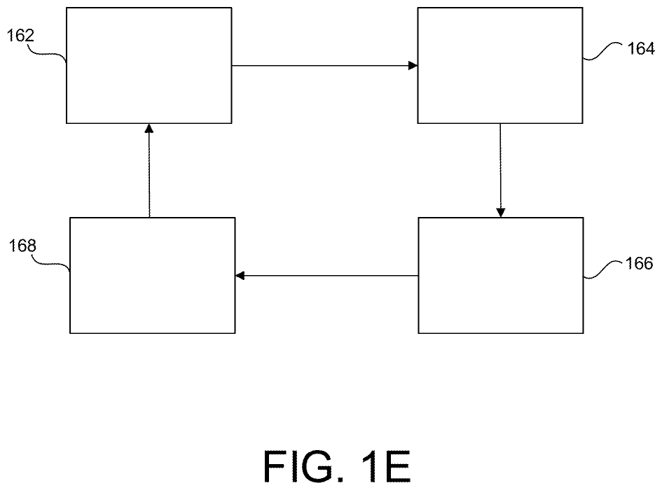

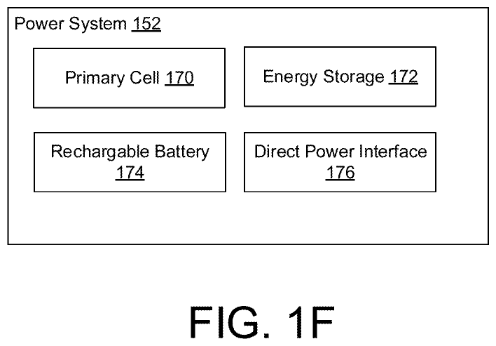

FIGS. 1A through 1F illustrate an inflatable penile prosthesis 100 having an electronic pump assembly 106 that can improve the performance of the inflatable penile prosthesis 100 according to an aspect. The inflatable penile prosthesis 100 includes a fluid reservoir 102, an inflatable member 104, and an electronic pump assembly 106 configured to transfer fluid between the fluid reservoir 102 and the inflatable member 104. The inflatable member 104 may be implanted into the corpus cavernosum of the user, and the fluid reservoir 102 may be implanted in the abdomen or pelvic cavity of the user (e.g., the fluid reservoir 102 may be implanted in the lower portion of the user's abdominal cavity or the upper portion of the user's pelvic cavity). In some examples, at least a portion of the electronic pump assembly 106 may be implemented in the patient's body.

The inflatable member 104 may be capable of expanding upon the injection of fluid into a cavity of the inflatable member 104. For instance, upon injection of the fluid into the inflatable member 104, the inflatable member 104 may increase its length and/or width, as well as increase its rigidity. In some examples, the inflatable member 104 includes a pair of inflatable cylinders or at least two cylinders, e.g., a first cylinder member and a second cylinder member. The volumetric capacity of the inflatable member 104 may depend on the size of the inflatable cylinders. The volume of fluid in each cylinder may vary from about 10 milliliters in smaller cylinders and to about 70 milliliters in larger sizes. In some examples, the first cylinder member may be larger than the second cylinder member. In some examples, the first cylinder member may have the same size as the second cylinder member.

The fluid reservoir 102 houses fluid that is used to inflate the inflatable member 104. The volumetric capacity of the fluid reservoir 102 may vary depending on the size of the inflatable penile prosthesis 100. The volumetric capacity of the fluid reservoir 102 may be up to 150 cubic centimeters. In some examples, the fluid reservoir 102 is constructed from the same material as the inflatable member 104. In some examples, the fluid reservoir 102 is constructed from a different material than the inflatable member 104. In some examples, the fluid reservoir 102 contains a larger volume of fluid than the inflatable member 104. In some examples, the fluid reservoir 102 is (or includes) a pressure-regulating flexible member. In some examples, the pressure-regulating flexible member may cause the inflatable member 104 to be partially inflated without activating any of the pumps 120. In some examples, the pressure-regulating flexible member includes an expandable balloon.

The inflatable penile prosthesis 100 may include a first conduit connector 103 and a second conduit connector 105. Each of the first conduit connector 103 and the second conduit connector 105 may define a lumen configured to transfer the fluid to and from the electronic pump assembly 106. The first conduit connector 103 may be coupled to the electronic pump assembly 106 and the fluid reservoir 102 such that fluid can be transferred between the electronic pump assembly 106 and the fluid reservoir 102 via the first conduit connector 103. For example, the first conduit connector 103 may define a first lumen configured to transfer fluid between the electronic pump assembly 106 and the fluid reservoir 102. The first conduit connector 103 may include a single or multiple tube members for transferring the fluid between the electronic pump assembly 106 and the fluid reservoir 102.

The second conduit connector 105 may be coupled to the electronic pump assembly 106 and the inflatable member 104 such that fluid can be transferred between the electronic pump assembly 106 and the inflatable member 104 via the second conduit connector 105. For example, the second conduit connector 105 may define a second lumen configured to transfer fluid between the electronic pump assembly 106 and the inflatable member 104. The second conduit connector 105 may include a single or multiple tube members for transferring the fluid between the electronic pump assembly 106 and the inflatable member 104. In some examples, the first conduit connector 103 and the second conduit connector 105 may include a silicone rubber material. In some examples, the electronic pump assembly 106 may be directly connected to the fluid reservoir 102.

The electronic pump assembly 106 may automatically transfer fluid between the fluid reservoir 102 and the inflatable member 104 without the user manually operating a pump (e.g., squeezing and releasing a pump bulb). The electronic pump assembly 106 includes one or more pumps 120, one or more active valves 118, a pressure sensor 130 configured to monitor a pressure of the inflatable member 104, and a controller 114 configured to control the pump(s) 120 and the active valve 118. For example, the controller 114 may control the pump(s) 120 to pump fluid between the fluid reservoir 102 and the inflatable member 104. The controller 114 may control the active valve 118 to transition between an open position and a closed position. The pump(s) 120 is configured to transfer fluid (on demand) to the inflatable member 104 at relatively high pressure (e.g., up to approximately 20.0 PSI).

FIG. 1B illustrates an example of the controller 114. The controller 114 may include a printed circuit board assembly (PCBA) 140. The PCBA 140 may include a communication module 142 configured to receive and process (e.g., interpret) an external signal, an on-board sensor 144 configured to detect pressure readings from the pressure sensor 130, a processor 146, a memory device 148, one or more drivers 150 to drive the pump(s) 120 and/or the active valve(s) 118, and a power system 152. In some examples, the on-board sensor 144 includes an accelerometer configured to detect movement and/or position of the patient.

The communication module 142 may include one or more communication circuits configured to receive an external signal. In some examples, the communication module 142 is a Bluetooth communication circuit that can receive and process a Bluetooth signal. In some examples, the communication module 142 is an NFC communication circuit that can receive and process an NFC signal. In some examples, the communication module 142 is a magnetic communication circuit that can receive and process a signal from a magnetic sensing circuit. In some examples, the communication module 142 is a high radio frequency (RF) communication circuit that can receive and process a high frequency RF signal. In some examples, the communication module 142 is a low frequency inductive communication circuit that can receive and process a low frequency inductive signal. In some examples, the communication module 142 is an ultrasonic communication circuit that can receive and process an ultrasonic signal. In some examples, the communication module 142 is a capacitive communication circuit that can receive and process a capacitive-based signal. In some examples, the communication module 142 is an acoustic processing circuit that can receive and process an acoustic signal.

Based on the external signals and/or pressure readings, the processor 146 may determine that an inflation cycle (and/or deflation cycle) has been activated and control the pump(s) 120 and the active valve 118 via the one or more drivers 150 to transfer fluid between the fluid reservoir 102 and the inflatable member 104. The processor 146 may be any type of controller configured to control operations of the pump(s) 120 and the active valve(s) 118. The processor 146 may be formed in a substrate configured to execute one or more machine executable instructions or pieces of software, firmware, or a combination thereof. The processor 146 can be semiconductor-based—that is, the processors can include semiconductor material that can perform digital logic. The processor 146 may be communicatively coupled to the driver of active valve(s) 118, the pump(s) 120, and the pressure sensor(s) 130.

The memory device 148 may store information in a format that can be read and/or executed by the processor 146. The memory device 148 may store executable instructions that when executed by the processor 146 cause the processor 146 to perform certain operations discussed herein. The processor 146 may receive data via the pressure sensor(s) 130 and/or the external device 101 and control the active valve(s) 118 and/or the pump(s) 120 by transmitting control signals to the active valve(s) 118 and/or the pump(s) 120. The memory device 148 may store control parameters that can be set or modified by the user and/or physician using the external device 101. In some examples, the memory device 148 may store a maximum pressure threshold and/or a partial inflation threshold. A user or physician may update the control parameters using the external device 101, which can be communicated to the controller 114 and then updated in the memory device 148. In some examples, the controller 114 may store usage statistics in the memory device 148. The usage statistics may include one or more statistics on the usage of the inflatable penile prosthetic 100.

In some examples, as shown in FIG. 1C, the controller 114 includes a microcontroller 154. In some examples, as shown in FIG. 1D, the processor 146 includes a state machine 156. The state machine 156 may be implemented by one or more field-programmable gate arrays 158 and/or one or more application-specific integrated circuits 160. As shown in FIG. 1E, the state machine 156 may transition between a plurality of states such as a standby state 162 in which the inflatable member 104 is in a deflated state, an inflation state 164 in the inflatable member 104 is inflated to a target pressure, a hold state 166 in which pressure is held at the target pressure, and a deflation state 168 in which the inflatable member is deflated.

In some examples, as shown in FIG. 1F, the power system 152 includes a primary cell 170. In some examples, the power system 152 includes an energy storage 172. The energy storage 172 may include one or more capacitors. In some examples, the power system 152 includes a rechargeable battery 174. In some examples, the power system 152 includes direct power interface 176. In some examples, low frequency techniques (e.g., inductive power) may enable activation as well as charge or directly power the inflatable penile prosthesis 100. The external charger may be a portable device or a static device such as a flat encapsulated coil (e.g., on a seat of a chair or on a mattress). In some examples, NFC transmissions may enable activation as well as charge or directly power the inflatable penile prosthesis 100. In some examples, VHF and UHF transmissions may enable activations as well as charge or directly power the inflatable penile prosthesis 100. In some examples, capacitive coupling technology may enable activation as well as charge or directly power the inflatable penile prosthesis 100.

In some examples, the electronic pump assembly 106 includes an antenna 112 configured to wirelessly transmit (and receive) wireless signals to (and from) an external device 101 (or multiple external devices 101). The wireless signals may be Bluetooth signals, NFC signals, magnetic signals, high frequency RF signals, low frequency inductive signals, ultrasonic signals, and/or capacitive-based signals. In some examples, the user can control the inflation or deflation cycles without using an external device 101. For example, the electronic pump assembly 106 may include an acoustic sensor that can sense acoustic signals (e.g., tapping), which can institute an inflation cycle or deflation cycle. In some examples, the user can squeeze the inflatable member 104, where the pressure sensor 130 may detect a pressure spike in the inflatable member 104, which may activate the inflation cycle.

The external device 101 may be any type of component that can communicate with the electronic pump assembly 106. The external device 101 may be a computer, custom device, smartphone, tablet, pendant, key fob, etc. In examples, the external device 101 includes one or more devices associated with the user of the inflatable penile prosthesis and one or more devices associated with a physician. The external device 101 may be an NFC device, a Bluetooth device, a magnetic device configured to generate a magnetic field, a radio frequency (RF) device configured to generate high frequency signals, an inductive device configured to generate low frequency inductive signals, an ultrasonic device configured to generate ultrasonic signals, or a capacitive device configured to generate a capacitance when placed in contact to a plate on the electronic pump assembly.

A user may use the external device 101 to control the inflatable penile prosthesis 100. The user may use the external device 101 to inflate or deflate the inflatable member 104. For example, in response to the user activating an inflation cycle using the external device 101, the external device 101 may transmit a wireless signal to the electronic pump assembly 106 to initiate the inflation cycle (received via the antenna 112), where the controller 114 may control the active valve(s) 118 and the pump(s) 120 to inflate the inflatable member 104 to a target inflation pressure. In some examples, the controller 114 may cause the active valve to a closed position and activate the pump(s) to move fluid from the fluid reservoir 102 to the inflatable member 104. The controller 114 may actuate the pump(s) 120 according to a pump frequency. In some examples, the pumping architecture is designed such that audio frequencies are minimized or avoided. The frequency range of 50 hz to 19 khz can be perceived by humans.

In response to the user activating a deflation cycle using the external device 101, the external device 101 may transmit a wireless signal to the electronic pump assembly 106 to initiate the deflation cycle (received via the antenna 112), where the controller 114 may control the active valve(s) 118 (and, in some examples, one or more of the pumps 120) to transfer fluid from the inflatable member 104 to the fluid reservoir 102. For example, the controller 114 may control the active valve 118 to move to the open position to allow fluid to transfer from the inflatable member 104 to the fluid reservoir 102. In some examples, the controller 114 may control one or more pumps 120 to further move the fluid from the inflatable member 104 to the fluid reservoir 102 during the deflation cycle. In some examples, during the deflation cycle, fluid is transferred back until the pressure in the inflatable member 104 reaches a partial inflation threshold. In some examples, the controller 114 may automatically determine to initiate a deflation cycle, which causes the controller 114 to control the active valve(s) 118 (and, in some examples, the pump(s) 120) to transfer fluid back to the fluid reservoir 102.

In some examples, the electronic pump assembly 106 includes a single pump 120 such as pump 120-1. The pump 120-1 may be disposed in parallel with the active valve 118. In some examples, the electronic pump assembly 106 includes multiple pumps 120. For example, the pumps 120 may include pump 120-1 and pump 120-2. In some examples, the pump 120-1 is disposed in a fluid passageway 125 that is used to fill the inflatable member 104 (e.g., during the inflation cycle). In some examples, the pump 120-2 is disposed in a parallel fluid passageway 127 that is used to fill the inflatable member 104 (e.g., during the inflation cycle). In some examples, the pump 120-2 is disposed in parallel with the pump 120-1. The pump 120-1 may transfer fluid according to a first flow rate, and the pump 120-1 may transfer fluid according to a second flow rate. In some examples, the first flow rate is substantially the same as the second flow rate. In some examples, the first flow rate is different from the second flow rate.

In some examples, the pumps 120 may include a pump disposed in series with the active valve 118. The pump may transfer fluid from the inflatable member 104 to the fluid reservoir 102 (e.g., during a deflation cycle). For example, during a deflation cycle, the controller 114 may activate the active valve 118 to be in the open position and may activate the pump to transfer fluid from the inflatable member 104 to the fluid reservoir 102. In some examples, the pump may transfer fluid according to a third flow rate. In some examples, the third flow rate is less than the first flow rate and/or the second flow rate. In some examples, the electronic pump assembly 106 may include one or more series pumps 120 and one or more parallel pumps 120. In some examples, the pumps 120 may include one or more pumps 120 in series with one or more other pumps 120. For example, one or more pumps 120 may be in series with the pump 120-1. In some examples, one or more pumps 120 may be in series with the pump 120-2.

Each pump 120 is an electronically-controlled pump. Each pump 120 may be electronically-controlled by the controller 114. For example, each pump 120 may be connected to the controller 114 and may receive a signal to actuate a respective pump 120. A pump 120 may be unidirectional in which the pump 120 can transfer fluid from the fluid reservoir 102 to the inflatable member 104 (or from the inflatable member 104 to the fluid reservoir 102). In some examples, a pump 120 is bidirectional in which the pump 120 can transfer fluid from the fluid reservoir 102 to the inflatable member 104 and from the inflatable member 104 to the fluid reservoir 102. In some examples, the pumps 120 are either unidirectional or bidirectional. In some examples, the pumps 120 include a combination of one or more unidirectional pumps and one or more bidirectional pumps.

In some examples, the pump 120 is an electromagnetic pump that moves the fluid between the fluid reservoir 102 and the inflatable member 104 using electromagnetism.

In some examples, the pump 120 is a piezoelectric pump. In some examples, a piezoelectric pump may be a diaphragm micropump that uses actuation of a diaphragm to drive a fluid. In some examples, a piezoelectric pump may include one or more piezo pumps (e.g., piezo elements), which may be implemented by a substrate layer (e.g., a single substrate layer) of high-voltage piezo elements or may be implemented by multiple substrate layers (e.g., stacked substrate layers) of low-voltage piezo elements. In some examples, the pump 120 includes a plurality of micro-pumps (e.g., piezoelectrically-driven micro-pumps) disposed on one or more substrates (e.g., wafer(s)). In some examples, the micro-pumps include a silicon-based material. In some examples, the micro-pumps include a metal (e.g., steel) based material. In some examples, the pump 120 is non-mechanical (e.g., without moving parts).

In some examples, in the case of multiple pumps 120, each pump 120 may be a pump of the same type (e.g., all pumps 120 are electromagnetic pumps or all pumps 120 are piezoelectric pumps). In some examples, one or more pumps 120 are different from one or more other pumps 120. For example, pumps 120 may include different types of piezoelectric pumps or the pumps 120 may include different types of electromagnetic pumps. The pump 120-1 may be a piezoelectric pump having a first number of micro-pumps, and the pump 120-2 may be a piezoelectric pump having a second number of micro-pumps

A pump 120 may include one or more passive check valves. The passive check valve(s) may assist with maintaining pressure in the inflatable member 104. In some examples, a pump 120 may include a single passive check valve. In some examples, the pump 120 may include multiple passive check valves such as two passive check valves or more than two passive check valves (e.g., disposed in series with each other). The passive check valve(s) of a respective pump 120 may not be directly controlled by the controller 114, but rather controlled based on the pressure between the inflatable member 104 and the fluid reservoir 102. The passive check valve(s) may transition between an open position (in which fluid is permitted to flow through the passive check valve(s)) and a closed position (in which fluid is prevented from flowing through the passive check valve(s)). In some examples, with respect to the pump 120-1 and the pump 120-2, the passive check valve(s) are forward biased to allow a passive flow through the passive check valve(s) in the direction from the fluid reservoir 102 to the inflatable member 104 while the passive check valve(s) are in the closed position in the flow direction from the inflatable member 104 to the fluid reservoir 102. In some examples, the passive check valve(s) transitions to the closed position in response to positive pressure between the inflatable member 104 and the fluid reservoir 102. In some examples, the passive check valve(s) transition to the open position in response to negative pressure between the inflatable member 104 and the fluid reservoir 102.

The active valve 118 may be an electronically-controlled valve. The active valve 118 may be electronically-controlled by the controller 114. For example, the active valve 118 may be connected to the controller 114 and may receive a signal to transition the active valve 118 between an open position in which the fluid flows through the active valve 118 and a closed position in which the fluid is prevented from flowing through the active valve 118. In some examples, the active valve 118 includes a diaphragm and a ring member (e.g., an O-ring). In some examples, in the closed position, the flow path is impeded by the interface between the diaphragm and the ring member. In some examples, in the open position, the diaphragm is related from the ring member (e.g., disposed a distance apart), which permits the fluid to flow through the active valve 118. An active valve 118 may be bidirectional. Each active valve 118 may be piezo or electromagnetic diaphragm actuated. In some examples, the active valve 118 is disposed in a fluid passageway 124 that is used to empty the inflatable member 104 (e.g., in the deflation cycle). In some examples, the active valve 118 may transition to the closed position to hold (e.g., substantially hold) the pressure in the inflatable member 104. In some examples, the active valve 118 may transition to the open position to transfer fluid back to the fluid reservoir 102, release pressure in the inflatable member 104 and/or allow a flow back to the inflatable member 104. In some examples, the active valve 118 may be used to hold (e.g., substantially hold) the partial inflation pressure.

In some examples, the electronic pump assembly 106 includes a single active valve 118. In some examples, the electronic pump assembly 106 includes multiple active valves 118. In some examples, one or more additional active valves 118 may be in series with the pump 120-1 and/or the pump 120-2. In some examples, the electronic pump assembly 106 does not include an active valve 118. In some examples, one or more of the pumps 120 may operate as a valve, therefore avoiding the use of an active valve 118. In some examples, an additional active valve 118 (e.g., a series active valve 118) may be disposed in a fluid pathway portion that is connected to the fluid reservoir 102. In some examples, an additional active valve 118 (e.g., a series active valve 118) may be disposed in a fluid pathway portion that is connected to the inflatable member 104. These additional active valves 118 may reduce leakage when at maximum inflation pressure or at partial inflation pressure.

The electronic pump assembly 106 may include one or more pressure sensors 130 configured to sense the pressure of the inflatable penile prosthesis 100. In some examples, the electronic pump assembly 106 includes a single pressure sensor 130. In some examples, the electronic pump assembly 106 may include multiple pressure sensors 130. The pressure sensor 130 is configured to measure the pressure of the inflatable member 104. The controller 114 may receive pressure readings from the pressure sensor 130 according to a pressure sensing rate. Each pressure reading may indicate the pressure of the inflatable member 104 at a time of the pressure reading. In some examples, the pressure sensor 130 is configured to measure the pressure of the reservoir 102. The pressure readings may be used to control the active valve(s) 118 to prevent (or reduce) transfer of fluid to the inflatable member 104.

The electronic pump assembly 106 may include a hermetic enclosure 108 that encloses the components of the electronic pump assembly 106. A hermetic enclosure 108 may be an air-tight (or substantially air-tight) container. The hermetic enclosure 108 may include one or more metal-based materials. In some examples, the hermetic enclosure 108 is a Titanium container. In some examples, the only material in contact with the patient is Titanium. In some examples, the hermetic enclosure 108 includes a non-metal-based material such as ceramic. In some examples, the hermetic enclosure 108 defines a feedthrough 136 (e.g., a hermetic feedthrough, an electrical feedthrough, a feedthrough connector, etc.) to receive/transmit wireless signals from/to the external device 101. In some examples, the feedthrough 136 includes a metal-based material and an insulator-based material (e.g., ceramic).

The electronic pump assembly 106 may include a hermetic fluid chamber 110 disposed inside of the hermetic enclosure 108. The hermetic fluid chamber 110 may be a separate air-tight (or substantially air-tight) container that is within the hermetic enclosure 108. The hermetic fluid chamber 110 may include one or more metal-based materials. In some examples, the hermetic fluid chamber 110 is a Titanium container. In some examples, the hermetic fluid chamber 110 includes one or more non-metal-based materials (e.g., ceramic). In some examples, a portion of the hermetic fluid chamber 110 is a metal-based material (e.g., Titanium) and a portion of the hermetic fluid chamber 110 is a non-metal-based material (e.g., ceramic). The hermetic fluid chamber 110 may isolate the fluid from the electronics. In other words, the electronics section may be isolated (e.g., completely isolated) from the fluid via the hermetic fluid chamber 110. The hermetic fluid chamber 110 may be fluidly connected to the fluid reservoir 102 and the inflatable member 104. The hermetic fluid chamber 110 may include the active valve(s) 118, the pump(s) 120, and the pressure sensor(s) 130. In some examples, the hermetic fluid chamber 110 defines a feedthrough 138 (e.g., a hermetic feedthrough, an electrical feedthrough, a feedthrough connector, etc.) to the controller 114 to receive/transmit signals from/to the controller 114. In some examples, the hermetic fluid chamber 110 disposed within the hermetic enclosure 108 creates a double hermetic system. In some examples, the electronic pump assembly 106 includes only one hermetic enclosure (e.g., the hermetic enclosure 108).

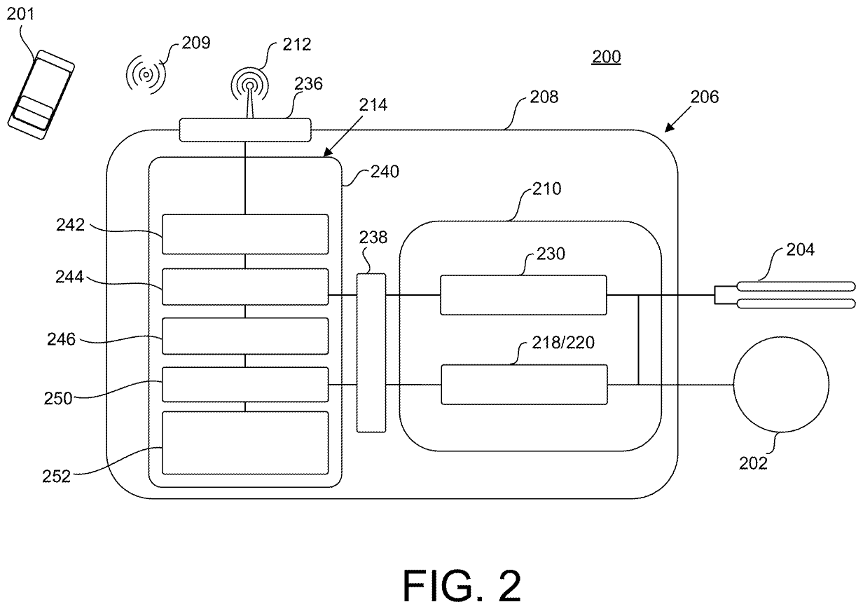

FIG. 2 illustrates an inflatable penile prosthesis 200 with an electronic pump assembly 206 configured to communicate with an external device 201 (e.g., a Bluetooth device) using a Bluetooth wireless connection according to an aspect. The inflatable penile prosthesis 200 may be an example of the inflatable penile prosthesis 100 of FIGS. 1A through 1F and may include any of the details discussed with reference to those figures.

A user may use the external device 201 to cause the electronic pump assembly 206 to inflate or deflate the inflatable member 204. For example, the user may operate the external device 201 to generate a Bluetooth signal 209, which can activate the inflation cycle. In some examples, the user may operate the external device 201 to generate a Bluetooth signal 209, which can activate the deflation cycle. Bluetooth is a short-range wireless standard utilized for exchanging data between devices over short distances using UHF radio waves in the ISM bands from 2.402 GHz to 2.48 GHz. Bluetooth low energy (BLE) may be suited to implantable devices especially where primary cells are used. A Bluetooth device (e.g., external device 201) such as a smart phone or a custom designed controller may be used to control the inflatable penile prosthesis 200 from outside the body.

The electronic pump assembly 206 is configured to transfer fluid between a fluid reservoir 202 and an inflatable member 204. The electronic pump assembly 206 includes a hermetic enclosure 208, an antenna 212, and a feedthrough 236. The electronic pump assembly 206 includes a controller 214 included within the hermetic enclosure 208. The antenna 212 may receive (and transmit) Bluetooth signals 209 from/to a Bluetooth device 201. The antenna 212 is connected to the controller 214 via the feedthrough 236.

The controller 214 includes a printed circuit board assembly 240 having a communication module 242 (e.g., a Bluetooth transceiver circuit), an on-board sensor 244, a processor 246 (with memory), one or more drivers 250, and a power system 252. In some examples, the processor 246 is a microprocessor. In some examples, the processor 246 is a state machine implemented by a field-programmable gate array and/or application-specific integrated circuit. The power system 252 may include a primary cell. In some examples, the power system 252 includes an energy storage (e.g., one or more capacitors). In some examples, the power system 252 includes a rechargeable battery. In some examples, the power system 252 includes a direct power interface.

The electronic pump assembly 206 includes a hermetic fluid chamber 210 disposed within the hermetic enclosure 208. The hermetic fluid chamber 210 includes a pressure sensor 230 configured to monitor the pressure of the inflatable member 204, one or more active valves 218, and one or more pumps 220. The on-board sensor 244 is connected to the pressure sensor 230 via a feedthrough 238 so that the controller 214 can receive the pressure readings from the pressure sensor 230. The active valve(s) 218 and the pumps 220 are connected to the driver(s) 250 via the feedthrough 238 to receive signals to activate/deactivate the pump(s) 220 and/or open or close the active valve(s) 218. The electronics section may be isolated (e.g., completely isolated) from the fluid via the hermetic fluid chamber 210.

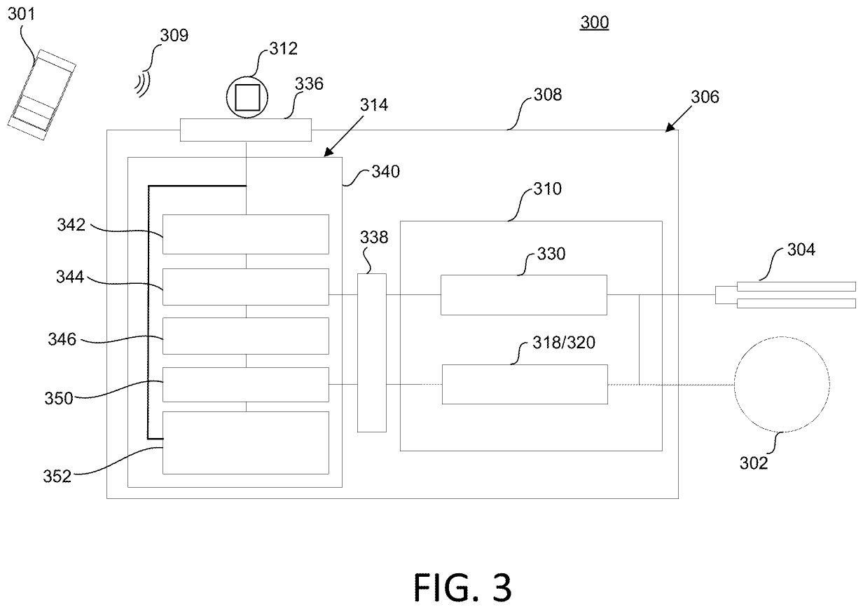

FIG. 3 illustrates an inflatable penile prosthesis 300 with an electronic pump assembly 306 configured to communicate with an external device 301 (e.g., NFC device) using an NFC wireless connection according to an aspect. The inflatable penile prosthesis 300 may be an example of the inflatable penile prosthesis 100 of FIGS. 1A through 1F and may include any of the details discussed with reference to those figures.

A user may use the external device 301 to cause the electronic pump assembly 306 to inflate or deflate the inflatable member 304. For example, the user may operate the external device 301 to generate an NFC signal 309, which can activate the inflation cycle. In some examples, the user may operate the external device 301 to generate an NFC signal 309, which can activate the deflation cycle.

Near-Field Communication (NFC) is a set of communication protocols for communication between two electronic devices over a distance of up to 4 cm less such as used in contactless payment systems. An NFC device (e.g., external device 301) such as a smart phone or a custom designed controller may be used to control the implantable device from outside the body.

The electronic pump assembly 306 is configured to transfer fluid between a fluid reservoir 302 and an inflatable member 304. The electronic pump assembly 306 includes a hermetic enclosure 308, an antenna 312, and a feedthrough 336. The electronic pump assembly 306 includes a controller 314 included within the hermetic enclosure 308. The antenna 312 may receive (and transmit) NFC signals 309 from/to an NFC device 301. The antenna 312 is connected to the controller 314 via the feedthrough 336.

The controller 314 includes a printed circuit board assembly 340 having a communication module 342 (e.g., an NFC transceiver circuit), an on-board sensor 344, a processor 346 (with memory), one or more drivers 350, and a power system 352. In some examples, the processor 346 is a microprocessor. In some examples, the processor 346 is a state machine implemented by a field-programmable gate array and/or application-specific integrated circuit. The power system 352 may include a primary cell. In some examples, the power system 352 includes an energy storage (e.g., one or more capacitors). In some examples, the power system 352 includes a rechargeable battery. In some examples, the power system 352 includes a direct power interface.

The electronic pump assembly 306 includes a hermetic fluid chamber 310 disposed within the hermetic enclosure 308. The hermetic fluid chamber 310 includes a pressure sensor 330 configured to monitor the pressure of the inflatable member 304, one or more active valves 318, and one or more pumps 320. The on-board sensor 344 is connected to the pressure sensor 330 via a feedthrough 338 so that the controller 314 can receive the pressure readings from the pressure sensor 330. The active valve(s) 318 and the pumps 320 are connected to the driver(s) 350 via the feedthrough 338 to receive signals to activate/deactivate the pump(s) 320 and/or open or close the active valve(s) 318. The electronics section may be isolated (e.g., completely isolated) from the fluid via the hermetic fluid chamber 310.

In some examples, NFC transmissions may enable activation as well as charge or directly power the electronic pump assembly 306. In some examples, the antenna 312 includes one or more directional antennas. Charging may be accomplished by routing the NFC transmissions to the power system 352 (e.g., the rechargeable battery, storage device).

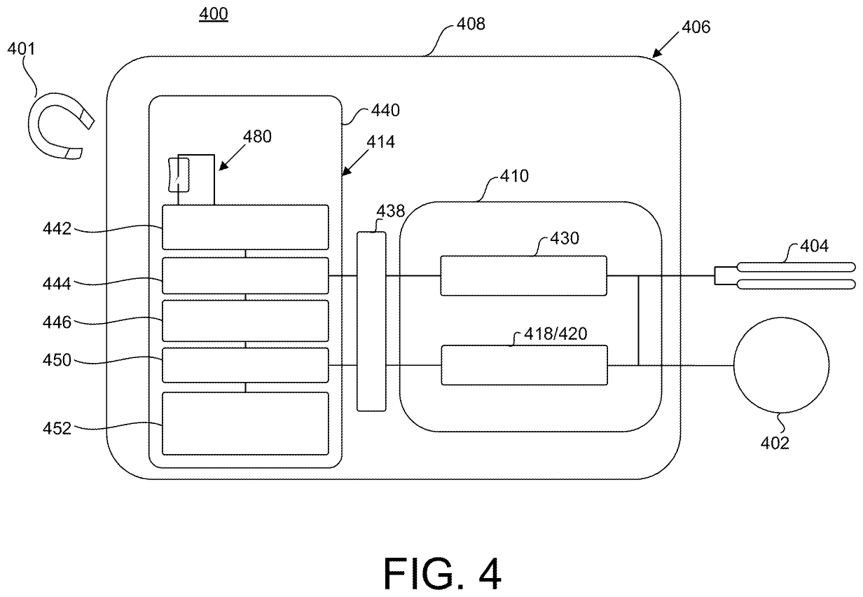

FIG. 4 illustrates an inflatable penile prosthesis 400 with an electronic pump assembly 406 configured to communicate with an external device 401 using a magnetic field according to an aspect. The inflatable penile prosthesis 400 may be an example of the inflatable penile prosthesis 100 of FIGS. 1A through 1F and may include any of the details discussed with reference to those figures.

A user may use the external device 401 to cause the electronic pump assembly 406 to inflate or deflate the inflatable member 404. For example, a magnet may be used by the patient to institute an inflation cycle or deflation cycle. The magnet may be supplied to the patient, and the electronic pump assembly 406 includes a magnet sensing switch 480 configured to sense the presence of a magnetic field.

The electronic pump assembly 406 is configured to transfer fluid between a fluid reservoir 402 and an inflatable member 404. The electronic pump assembly 406 includes a hermetic enclosure 408. The electronic pump assembly 406 includes a controller 414 included within the hermetic enclosure 408.

The controller 414 includes a printed circuit board assembly 440 having a magnet sensing switch 480 configured to close in the presence of a magnetic field provided by the external device 401, a communication module 442 (e.g., that receives a voltage from the magnet sensing switch 480), an on-board sensor 444, a processor 446 (with memory), one or more drivers 450, and a power system 452. In some examples, the processor 446 is a microprocessor. In some examples, the processor 446 is a state machine implemented by a field-programmable gate array and/or application-specific integrated circuit. The power system 452 may include a primary cell. In some examples, the power system 452 includes an energy storage (e.g., one or more capacitors). In some examples, the power system 452 includes a rechargeable battery. In some examples, the power system 452 includes a direct power interface.

The electronic pump assembly 406 includes a hermetic fluid chamber 410 disposed within the hermetic enclosure 408. The hermetic fluid chamber 410 includes a pressure sensor 430 configured to monitor the pressure of the inflatable member 404, one or more active valves 418, and one or more pumps 420. The on-board sensor 444 is connected to the pressure sensor 430 via a feedthrough 438 so that the controller 414 can receive the pressure readings from the pressure sensor 430. The active valve(s) 418 and the pumps 420 are connected to the driver(s) 450 via the feedthrough 438 to receive signals to activate/deactivate the pump(s) 420 and/or open or close the active valve(s) 418. The electronics section may be isolated (e.g., completely isolated) from the fluid via the hermetic fluid chamber 410.

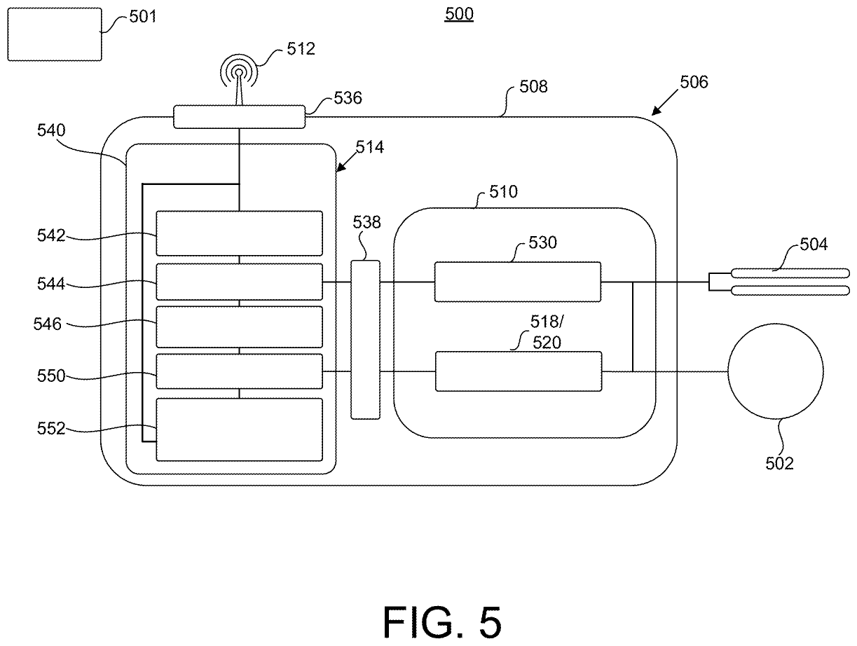

FIG. 5 illustrates an inflatable penile prosthesis 500 with an electronic pump assembly 506 configured to communicate with an external device 501 using high frequency radio signals according to an aspect. The inflatable penile prosthesis 500 may be an example of the inflatable penile prosthesis 100 of FIGS. 1A through 1F and may include any of the details discussed with reference to those figures.

A user may use the external device 501 to cause the electronic pump assembly 506 to inflate or deflate the inflatable member 504. For example, the user may operate the external device 401 to generate a high frequency radio signal, which can activate the inflation cycle. In some examples, the user may operate the external device 301 to generate a high frequency radio signal, which can activate the deflation cycle.

High frequency transmissions may be used to activate the inflatable penile prosthesis 500. Frequencies may range from kHz to GHz ranges. Inductive coupling, resonant inductive coupling and microwave may be utilized in mostly near and mid-field operations. In some examples, the external device 501 includes a custom RF controller, which may be used to control the inflatable penile prosthesis 500 from outside the body.

The electronic pump assembly 506 is configured to transfer fluid between a fluid reservoir 502 and an inflatable member 504. The electronic pump assembly 506 includes a hermetic enclosure 508, an antenna 512, and a feedthrough 536. The electronic pump assembly 506 includes a controller 514 included within the hermetic enclosure 508. The antenna 512 may receive (and transmit) RF signals from/to the external device 501. The antenna 512 is connected to the controller 514 via the feedthrough 536.

The controller 514 includes a printed circuit board assembly 540 having a communication module 542 (e.g., an RF transceiver circuit), an on-board sensor 544, a processor 546 (with memory), one or more drivers 550, and a power system 552. In some examples, the processor 546 is a microprocessor. In some examples, the processor 546 is a state machine implemented by a field-programmable gate array and/or application-specific integrated circuit. The power system 552 may include a primary cell. In some examples, the power system 552 includes an energy storage (e.g., one or more capacitors). In some examples, the power system 552 includes a rechargeable battery. In some examples, the power system 552 includes a direct power interface.

The electronic pump assembly 506 includes a hermetic fluid chamber 510 disposed within the hermetic enclosure 508. The hermetic fluid chamber 510 includes a pressure sensor 530 configured to monitor the pressure of the inflatable member 504, one or more active valves 518, and one or more pumps 520. The on-board sensor 544 is connected to the pressure sensor 530 via a feedthrough 538 so that the controller 514 can receive the pressure readings from the pressure sensor 530. The active valve(s) 518 and the pumps 520 are connected to the driver(s) 550 via the feedthrough 538 to receive signals to activate/deactivate the pump(s) 520 and/or open or close the active valve(s) 518. The electronics section may be isolated (e.g., completely isolated) from the fluid via the hermetic fluid chamber 510.

VHF and UHF transmissions may enable activation as well as charge or directly power the inflatable penile prosthesis 500. Frequencies are typically in the MHz to GHz ranges and incorporate resonant or non-resonant modes. Near and mid field techniques may be incorporated. In some examples, the antenna 512 includes one or more directional antennas to improve power transfer.

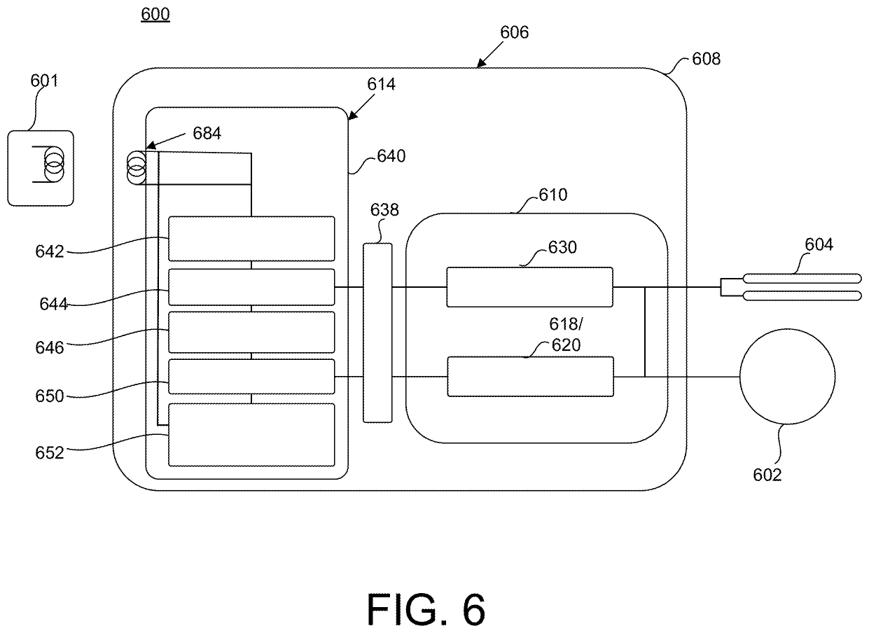

FIG. 6 illustrates an inflatable penile prosthesis 600 with an electronic pump assembly 606 configured to communicate with an external device 601 using low frequency inductive signals according to an aspect. The inflatable penile prosthesis 600 may be an example of the inflatable penile prosthesis 100 of FIGS. 1A through 1F and may include any of the details discussed with reference to those figures.

A user may use the external device 601 to cause the electronic pump assembly 606 to inflate or deflate the inflatable member 604. In some examples, the external device 601 includes an inductive controller. The user may operate the external device 601 to generate an inductive signal, which can activate the inflation cycle. In some examples, the user may operate the external device 601 to generate an inductive signal, which can activate the deflation cycle. Low frequency techniques may activate the inflatable penile prosthesis 600. Frequencies are typically in the kHz ranges and incorporate resonant or non-resonant modes.

The electronic pump assembly 606 is configured to transfer fluid between a fluid reservoir 602 and an inflatable member 604. The electronic pump assembly 606 includes a hermetic enclosure 608. The electronic pump assembly 606 includes a controller 614 included within the hermetic enclosure 608.

The controller 614 includes a printed circuit board assembly 640 having an inductive antenna, a communication module 642 (e.g., a transceiver circuit), an on-board sensor 644, a processor 646 (with memory), one or more drivers 650, and a power system 652. In some examples, the processor 646 is a microprocessor. In some examples, the processor 646 is a state machine implemented by a field-programmable gate array and/or application-specific integrated circuit. The power system 652 may include a primary cell. In some examples, the power system 652 includes an energy storage (e.g., one or more capacitors). In some examples, the power system 652 includes a rechargeable battery. In some examples, the power system 652 includes a direct power interface.

The electronic pump assembly 606 includes a hermetic fluid chamber 610 disposed within the hermetic enclosure 608. The hermetic fluid chamber 610 includes a pressure sensor 630 configured to monitor the pressure of the inflatable member 604, one or more active valves 618, and one or more pumps 620. The on-board sensor 644 is connected to the pressure sensor 630 via a feedthrough 638 so that the controller 614 can receive the pressure readings from the pressure sensor 630. The active valve(s) 618 and the pumps 620 are connected to the driver(s) 650 via the feedthrough 638 to receive signals to activate/deactivate the pump(s) 620 and/or open or close the active valve(s) 618. The electronics section may be isolated (e.g., completely isolated) from the fluid via the hermetic fluid chamber 610.

Low frequency techniques may enable activation as well as charge or directly power the inflatable penile prosthesis 600. Frequencies are typically in the kHz ranges and incorporate resonant or non-resonant modes.

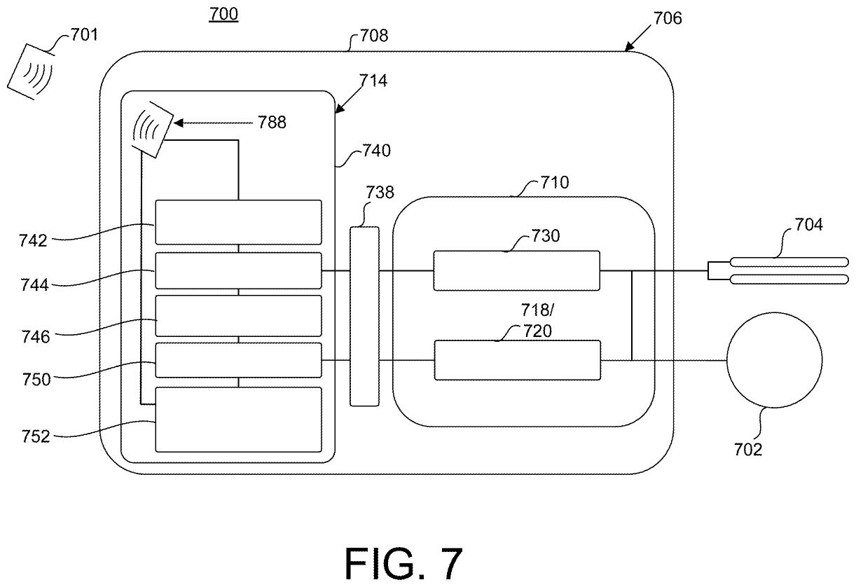

FIG. 7 illustrates an inflatable penile prosthesis 700 with an electronic pump assembly 706 configured to communicate with an external device 701 using ultrasonic signals according to an aspect. The inflatable penile prosthesis 700 may be an example of the inflatable penile prosthesis 100 of FIGS. 1A through IF and may include any of the details discussed with reference to those figures.

A user may use the external device 701 to cause the electronic pump assembly 706 to inflate or deflate the inflatable member 704. In some examples, the external device 701 includes an ultrasonic transceiver configured to communicate with an ultrasonic antenna 788 on a controller 714 of the electronic pump assembly 706. The user may operate the external device 701 to generate an ultrasonic signal, which can activate the inflation cycle. In some examples, the user may operate the external device 701 to generate an ultrasonic signal which can activate the deflation cycle. Ultrasonic transmissions may be used to activate the inflatable penile prosthesis 700 using piezoelectric technologies.

The electronic pump assembly 706 is configured to transfer fluid between a fluid reservoir 702 and an inflatable member 704. The electronic pump assembly 706 includes a hermetic enclosure 708. The electronic pump assembly 706 includes a controller 714 included within the hermetic enclosure 708.

The controller 714 includes a printed circuit board assembly 740 having an ultrasonic antenna 788, a communication module 742 (e.g., a transceiver circuit), an on-board sensor 744, a processor 746 (with memory), one or more drivers 750, and a power system 752. In some examples, the processor 746 is a microprocessor. In some examples, the processor 746 is a state machine implemented by a field-programmable gate array and/or application-specific integrated circuit. The power system 752 may include a primary cell. In some examples, the power system 752 includes an energy storage (e.g., one or more capacitors). In some examples, the power system 752 includes a rechargeable battery. In some examples, the power system 752 includes a direct power interface.

The electronic pump assembly 706 includes a hermetic fluid chamber 710 disposed within the hermetic enclosure 708. The hermetic fluid chamber 710 includes a pressure sensor 730 configured to monitor the pressure of the inflatable member 704, one or more active valves 718, and one or more pumps 720. The on-board sensor 744 is connected to the pressure sensor 730 via a feedthrough 738 so that the controller 714 can receive the pressure readings from the pressure sensor 730. The active valve(s) 718 and the pumps 720 are connected to the driver(s) 750 via the feedthrough 738 to receive signals to activate/deactivate the pump(s) 720 and/or open or close the active valve(s) 718. The electronics section may be isolated (e.g., completely isolated) from the fluid via the hermetic fluid chamber 710. In some examples, ultrasonic techniques can be used to remotely power the device (e.g., directly power the device), and/or remotely recharge its rechargeable cell or storage device.

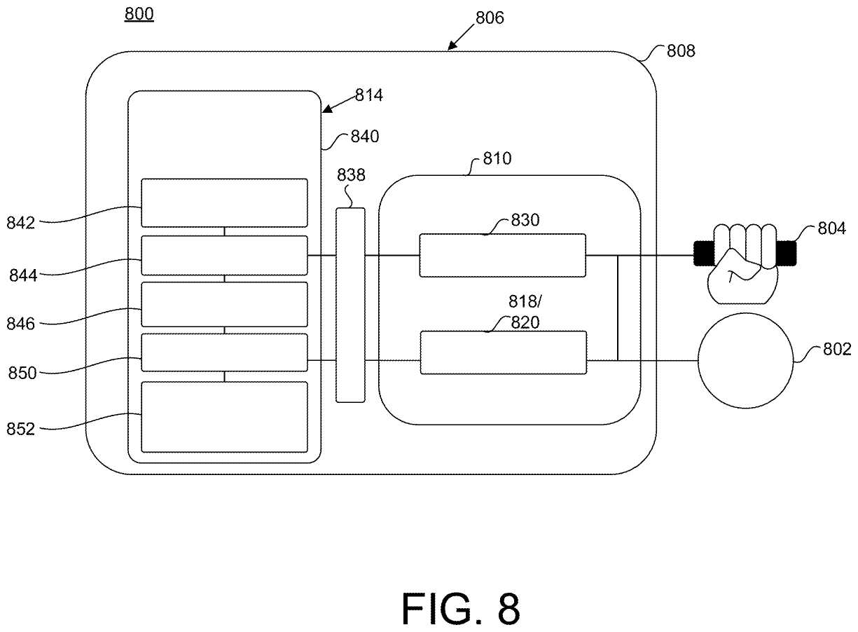

FIG. 8 illustrates an inflatable penile prosthesis 800 with an electronic pump assembly 806 configured to activate an inflation or deflation cycle based on a gripping signal sensed by a pressure sensor 830 according to an aspect. The inflatable penile prosthesis 800 may be an example of the inflatable penile prosthesis 100 of FIGS. 1A through 1F and may include any of the details discussed with reference to those figures.

A grasp action on the inflatable member 804 may cause compression on the inflatable member 804 to trigger an inflation cycle. In some examples, the inflatable penile prosthesis 800 may not use an external device. When deflation is required, the controller 814 may detect a series of grasp actions in order to avoid false signals. Deactivation could optionally also be triggered by another external device or a timer. The pressure sensor 830 detects the signal and it is processed by on-board sensor 844.

The electronic pump assembly 806 is configured to transfer fluid between a fluid reservoir 802 and an inflatable member 804. The electronic pump assembly 806 includes a hermetic enclosure 808. The electronic pump assembly 806 includes a controller 814 included within the hermetic enclosure 808.

The controller 814 includes a printed circuit board assembly 840 having a communication module 842 (e.g., a transceiver circuit), an on-board sensor 844, a processor 846 (with memory), one or more drivers 850, and a power system 852. In some examples, the processor 846 is a microprocessor. In some examples, the processor 846 is a state machine implemented by a field-programmable gate array and/or application-specific integrated circuit. The power system 852 may include a primary cell. In some examples, the power system 852 includes an energy storage (e.g., one or more capacitors). In some examples, the power system 852 includes a rechargeable battery. In some examples, the power system 852 includes a direct power interface.

The electronic pump assembly 806 includes a hermetic fluid chamber 810 disposed within the hermetic enclosure 808. The hermetic fluid chamber 810 includes a pressure sensor 830 configured to monitor the pressure of the inflatable member 804, one or more active valves 818, and one or more pumps 820. The on-board sensor 844 is connected to the pressure sensor 830 via a feedthrough 838 so that the controller 814 can receive the pressure readings from the pressure sensor 830. The active valve(s) 818 and the pumps 820 are connected to the driver(s) 850 via the feedthrough 838 to receive signals to activate/deactivate the pump(s) 820 and/or open or close the active valve(s) 818. The electronics section may be isolated (e.g., completely isolated) from the fluid via the hermetic fluid chamber 810.

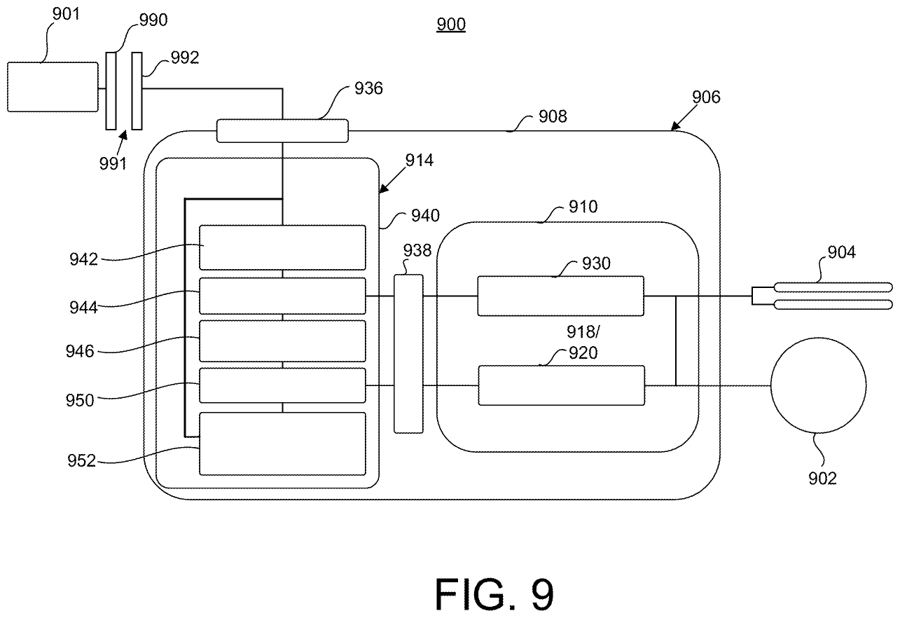

FIG. 9 illustrates an inflatable penile prosthesis 900 with an electronic pump assembly 906 configured to communicate with an external device 901 using a capacitive signal according to an aspect. The inflatable penile prosthesis 900 may be an example of the inflatable penile prosthesis 100 of FIGS. 1A through 1F and may include any of the details discussed with reference to those figures.

In some examples, the external device 901 is a capacitive device (e.g., having a first plate 990 of a capacitor 991), where the electronic pump assembly 906 includes a second plate 992 of the capacitor 991. For example, the first plate 990 is part of the external device 901 and the second plate 992 is implanted in the body of the patient. When the first plate 990 is placed in close proximity (e.g., within a threshold distance) to the second plate 992, a capacitive signal may be received by the controller 914 (via a feedthrough 936) to cause the inflatable member 904 to be inflated or deflated.

The electronic pump assembly 906 is configured to transfer fluid between a fluid reservoir 902 and an inflatable member 904. The electronic pump assembly 906 includes a hermetic enclosure 908, the first plate 992, and a feedthrough 936 (that allows the capacitive signal to be received at the controller 914). The electronic pump assembly 906 includes a controller 914 included within the hermetic enclosure 908.

The controller 914 includes a printed circuit board assembly 940 having a communication module 942 (e.g., a transceiver circuit), an on-board sensor 944, a processor 946 (with memory), one or more drivers 950, and a power system 952. In some examples, the processor 946 is a microprocessor. In some examples, the processor 946 is a state machine implemented by a field-programmable gate array and/or application-specific integrated circuit. The power system 952 may include a primary cell. In some examples, the power system 952 includes an energy storage (e.g., one or more capacitors). In some examples, the power system 952 includes a rechargeable battery. In some examples, the power system 952 includes a direct power interface.

The electronic pump assembly 906 includes a hermetic fluid chamber 910 disposed within the hermetic enclosure 908. The hermetic fluid chamber 910 includes a pressure sensor 930 configured to monitor the pressure of the inflatable member 904, one or more active valves 918, and one or more pumps 920. The on-board sensor 944 is connected to the pressure sensor 930 via a feedthrough 938 so that the controller 914 can receive the pressure readings from the pressure sensor 930. The active valve(s) 918 and the pumps 920 are connected to the driver(s) 950 via the feedthrough 938 to receive signals to activate/deactivate the pump(s) 920 and/or open or close the active valve(s) 918. The electronics section may be isolated (e.g., completely isolated) from the fluid via the hermetic fluid chamber 910.

Capacitive coupling technology may enable activation as well as charge or directly power the inflatable penile prosthesis 900. The external device 901 has a plate (e.g., first plate 990) that is placed adjacent to skin. The second plate 992 is implanted and may be a part of a shallow implant or a satellite transducer just under the skin.

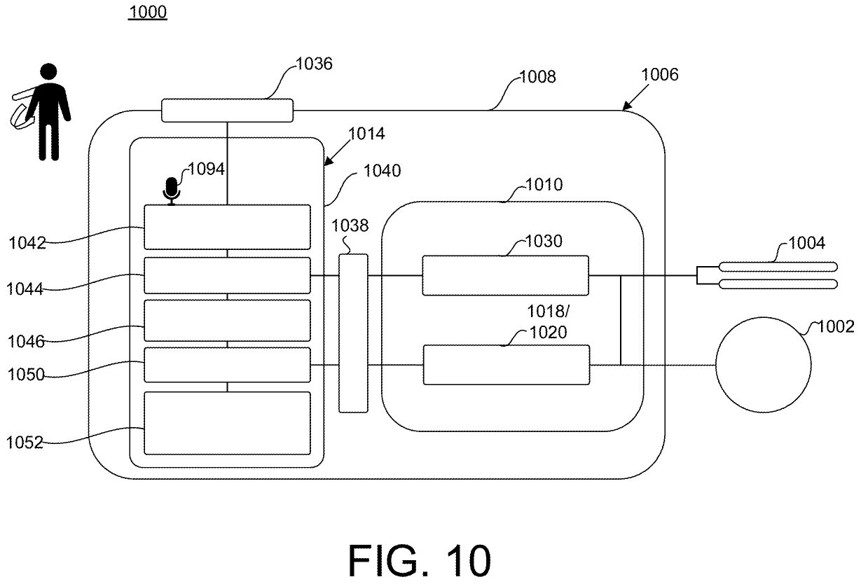

FIG. 10 illustrates an inflatable penile prosthesis 1000 with an electronic pump assembly 1006 configured to activate an inflation or deflation cycle using an acoustic signal according to an aspect. The inflatable penile prosthesis 1000 may be an example of the inflatable penile prosthesis 100 of FIGS. 1A through IF and may include any of the details discussed with reference to those figures. Tapping on the abdomen can be detected by an acoustic sensor 1094 to trigger inflation. In some examples, the acoustic signal may be used to trigger inflation or deflation.

The electronic pump assembly 1006 is configured to transfer fluid between a fluid reservoir 1002 and an inflatable member 1004. The electronic pump assembly 1006 includes a hermetic enclosure 1008, and a feedthrough 1036 (that allows the acoustic signal to be detected by an acoustic sensor 1094). In some examples, the acoustic sensor 1094 is disposed outside the electronic pump assembly 1006 (e.g., on the other side of the feedthrough 1036). The electronic pump assembly 1006 includes a controller 1014 included within the hermetic enclosure 1008.

The controller 1014 includes a printed circuit board assembly 1040 having an acoustic sensor 1094, a communication module 1042 (e.g., a transceiver circuit), an on-board sensor 1044, a processor 1046 (with memory), one or more drivers 1050, and a power system 1052. In some examples, the processor 1046 is a microprocessor. In some examples, the processor 1046 is a state machine implemented by a field-programmable gate array and/or application-specific integrated circuit. The power system 1052 may include a primary cell. In some examples, the power system 1052 includes an energy storage (e.g., one or more capacitors). In some examples, the power system 1052 includes a rechargeable battery. In some examples, the power system 1052 includes a direct power interface.

The electronic pump assembly 1006 includes a hermetic fluid chamber 1010 disposed within the hermetic enclosure 1008. The hermetic fluid chamber 1010 includes a pressure sensor 1030 configured to monitor the pressure of the inflatable member 1004, one or more active valves 1018, and one or more pumps 1020. The on-board sensor 1044 is connected to the pressure sensor 1030 via a feedthrough 1038 so that the controller 1014 can receive the pressure readings from the pressure sensor 1030. The active valve(s) 1018 and the pumps 1020 are connected to the driver(s) 1050 via the feedthrough 1038 to receive signals to activate/deactivate the pump(s) 1020 and/or open or close the active valve(s) 1018. The electronics section may be isolated (e.g., completely isolated) from the fluid via the hermetic fluid chamber 1010.

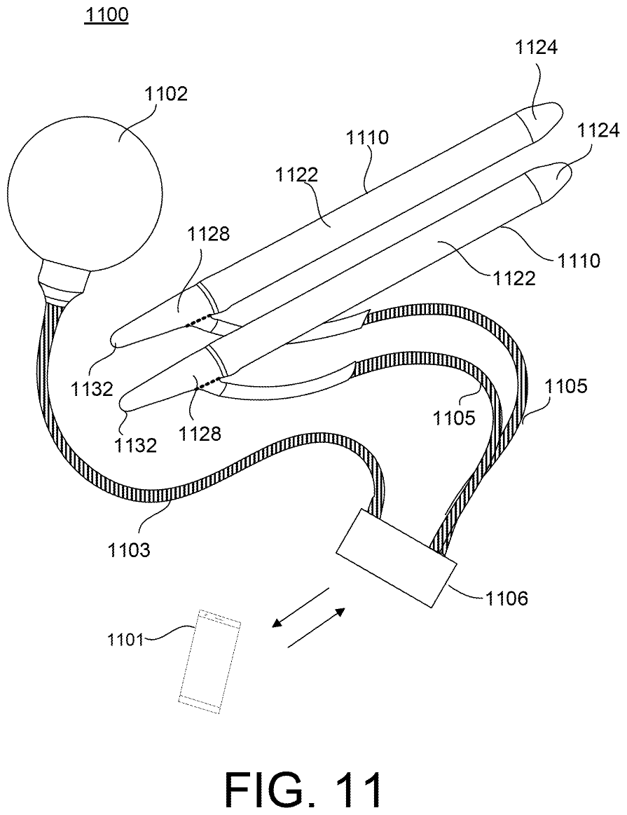

FIG. 11 schematically illustrates an inflatable penile prosthesis 1100 having an electronic pump assembly 1106 according to an aspect. The electronic pump assembly 1106 may include any of the features of the electronic pump assembly discussed with reference to the previous figures. The inflatable penile prosthesis 1100 may include a pair of inflatable cylinders 1110, and the inflatable cylinders 1110 are configured to be implanted in a penis. For example, one of the inflatable cylinders 1110 may be disposed on one side of the penis, and the other inflatable cylinder 1110 may be disposed on the other side of the penis. Each inflatable cylinder 1110 may include a first end portion 1124, a cavity or inflation chamber 1122, and a second end portion 1128 having a rear tip 1132.

At least a portion of the electronic pump assembly 1106 may be implanted in the patient's body. A pair of conduit connectors 1105 may attach the electronic pump assembly 1106 to the inflatable cylinders 1110 such that the electronic pump assembly 1106 is in fluid communication with the inflatable cylinders 1110. Also, the electronic pump assembly 1106 may be in fluid communication with a fluid reservoir 1102 via a conduit connector 1103. The fluid reservoir 1102 may be implanted into the user's abdomen. The inflation chamber 1122 of the inflatable cylinder 1110 may be disposed within the penis. The first end portion 1124 of the inflatable cylinder 1110 may be at least partially disposed within the crown portion of the penis. The second end portion 1128 may be implanted into the patient's pubic region PR with the rear tip 1132 proximate to the pubic bone PB.

In order to implant the inflatable cylinders 1110, the surgeon first prepares the patient. The surgeon often makes an incision in the penoscrotal region, e.g., where the base of the penis meets with the top of the scrotum. From the penoscrotal incision, the surgeon may dilate the patient's corpus cavernosum to prepare the patient to receive the inflatable cylinders 1110. The corpus cavernosum is one of two parallel columns of erectile tissue forming the dorsal part of the body of the penis, e.g., two slender columns that extend substantially the length of the penis. The surgeon will also dilate two regions of the pubic area to prepare the patient to receive the second end portion 1128. The surgeon may measure the length of the corpora cavernosum from the incision and the dilated region of the pubic area to determine an appropriate size of the inflatable cylinders 1110 to implant.

After the patient is prepared, the penile prosthesis 1100 is implanted into the patient. The tip of the first end portion 1124 of each inflatable cylinder 1110 may be attached to a suture. The other end of the suture may be attached to a needle member (e.g., Keith needle). The needle member is inserted into the incision and into the dilated corpus cavernosum. The needle member is then forced through the crown of the penis. The surgeon tugs on the suture to pull the inflatable cylinder 1110 into the corpus cavernosum. This is done for each inflatable cylinder 1110 of the pair. Once the inflation chamber 1122 is in place, the surgeon may remove the suture from the tip. The surgeon then inserts the second end portion 1128. The surgeon inserts the rear end of the inflatable cylinder 1110 into the incision and forces the second end portion 1128 toward the pubic bone PB until each inflatable cylinder 1110 is in place. However, the embodiments also encompass other various implant techniques.

A user may use an external device 1101 to control the inflatable penile prosthesis 1100. In some examples, the user may use the external device 1101 to inflate or deflate the inflatable cylinders 1110. For example, in response to the user activating an inflation cycle using the external device 1101, the external device 1101 may transmit a wireless signal to the electronic pump assembly 1106 to initiate the inflation cycle to transfer fluid from the fluid reservoir 1102 to the inflatable cylinders 1110. In some examples, in response to the user activating a deflation cycle using the external device 1101, the external device 1101 may transmit a wireless signal to the electronic pump assembly 1106 to initiate the deflation cycle to transfer fluid from the inflatable cylinders 1110 to the fluid reservoir 1102. In some examples, during the deflation cycle, fluid is transferred back until the pressure in the inflatable cylinders 1110 reaches a partial inflation pressure.

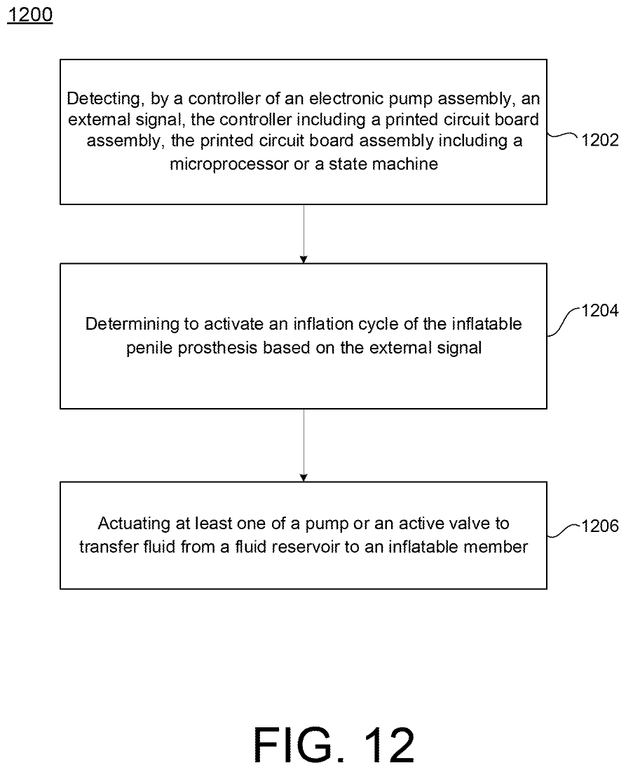

FIG. 12 illustrates a flow chart 1200 depicting example operations of a method of operating an electronic pump assembly of an inflatable penile prosthesis. The example operations of the flow chart 1200 may be performed by any of the inflatable penile prostheses and/or the electronic pump assemblies discussed herein.

Operation 1202 includes detecting, by a controller of an electronic pump assembly, an external signal, where the controller includes a printed circuit board assembly, and the printed circuit board assembly includes a microprocessor or a state machine. Operation 1204 includes determining to activate an inflation cycle of the inflatable penile prosthesis based on the external signal. Operation 1206 includes actuating at least one of a pump or an active valve to transfer fluid from a fluid reservoir to an inflatable member. In some examples, the method includes charging a battery of the electronic pump assembly based on wireless transmissions received via an antenna of the electronic pump assembly. In some examples, the battery can be charged based on inductive, ultrasonic, NFC, high frequency RF, or capacitive wireless transmissions.

Detailed embodiments are disclosed herein. However, it is understood that the disclosed embodiments are merely examples, which may be embodied in various forms. Therefore, specific structural and functional details disclosed herein are not to be interpreted as limiting, but merely as a basis for the claims and as a representative basis for teaching one skilled in the art to variously employ the embodiments in virtually any appropriately detailed structure. Further, the terms and phrases used herein are not intended to be limiting, but to provide an understandable description of the present disclosure.

The terms “a” or “an,” as used herein, are defined as one or more than one. The term “another,” as used herein, is defined as at least a second or more. The terms “including” and/or “having”, as used herein, are defined as comprising (i.e., open transition). The term “coupled” or “moveably coupled,” as used herein, is defined as connected, although not necessarily directly and mechanically.

In general, the embodiments are directed to bodily implants. The term patient or user may hereafter be used for a person who benefits from the medical device or the methods disclosed in the present disclosure. For example, the patient can be a person whose body is implanted with the medical device or the method disclosed for operating the medical device by the present disclosure. For example, in some embodiments, the patient may be a human.

While certain features of the described implementations have been illustrated as described herein, many modifications, substitutions, changes, and equivalents will now occur to those skilled in the art. It is, therefore, to be understood that the appended claims are intended to cover all such modifications and changes as fall within the scope of the embodiments.

What is claimed is:

1. An inflatable penile prosthesis comprising:
a fluid reservoir configured to hold fluid;
an inflatable member; and
an electronic pump assembly configured to transfer the fluid between the fluid reservoir and the inflatable member, the electronic pump assembly including:
a pump;
an active valve; and
a controller configured to receive an external signal to activate an inflation cycle and control at least one of the pump or the active valve to transfer the fluid to the inflatable member, the controller including a printed circuit board assembly, the printed circuit board assembly including a microprocessor or a state machine.

2. The inflatable penile prosthesis of claim 1, wherein the electronic pump assembly includes an antenna, the antenna configured to receive the external signal, the external signal including a Bluetooth signal.

3. The inflatable penile prosthesis of claim 1, wherein the electronic pump assembly includes an antenna, the antenna configured to receive the external signal, the external signal including a near-field communication (NFC) signal.

4. The inflatable penile prosthesis of claim 3, the printed circuit board assembly including a power source, wherein the power source is configured to be charged by the NFC signal.

5. The inflatable penile prosthesis of claim 1, wherein the printed circuit board assembly includes a magnetic sensing circuit, the controller configured to detect activation of the inflation cycle in response to the magnetic sensing circuit detecting a presence of a magnetic field.

6. The inflatable penile prosthesis of claim 1, wherein the electronic pump assembly includes an antenna, the antenna configured to receive the external signal, the external signal including a radio frequency (RF) signal.

7. The inflatable penile prosthesis of claim 1, wherein the electronic pump assembly includes an inductive antenna, the inductive antenna configured to receive the external signal, the external signal including an inductive signal.

8. The inflatable penile prosthesis of claim 1, wherein the electronic pump assembly includes an antenna, the antenna configured to receive the external signal, the external signal including an ultrasonic signal.

9. The inflatable penile prosthesis of claim 1, wherein the electronic pump assembly includes a pressure sensor, the controller configured to receive a pressure reading from the pressure sensor that indicates a pressure of the inflatable member, the controller configured to determine that the inflation cycle is activated in response to pressure exceeding a threshold level.

10. The inflatable penile prosthesis of claim 1, wherein the electronic pump assembly includes a first conductive plate, the controller configured to determine activation of the inflation cycle in response to a second conductive plate of an external device being placed within a threshold distance of the first conductive plate.

11. The inflatable penile prosthesis of claim 1, wherein the electronic pump assembly includes an acoustic sensor configured to receive an acoustic signal, the controller configured to determine activation of the inflation cycle in response to the acoustic signal.

12. An inflatable penile prosthesis comprising:
a fluid reservoir configured to hold fluid;
an inflatable member; and
an electronic pump assembly configured to transfer the fluid between the fluid reservoir and the inflatable member, the electronic pump assembly including:
a pump;
an active valve;
an antenna configured to receive an external signal;
a controller configured to determine to activate an inflation cycle based on the external signal and control at least one of the pump or the active valve to transfer the fluid to the inflatable member; and
a power source configured to power the controller, the power source configured to be recharged by wireless transmissions received via the antenna.

13. The inflatable penile prosthesis of claim 12, wherein the wireless transmissions include near-field communication (NFC) transmissions.

14. The inflatable penile prosthesis of claim 12, wherein the wireless transmissions include radio frequency (RF) transmissions.

15. The inflatable penile prosthesis of claim 12, wherein the wireless transmissions include capacitive or inductive transmissions.

16. The inflatable penile prosthesis of claim 12, wherein the controller includes a printed circuit board assembly, the printed circuit board assembly including a microprocessor configured to interpret the external signal.

17. The inflatable penile prosthesis of claim 12, wherein the controller includes a printed circuit board assembly, the printed circuit board assembly including a state machine configured to interpret the external signal.

18. The inflatable penile prosthesis of claim 17, wherein the state machine includes a field-programmable gate array or an application-specific integrated circuit.

19. A method of controlling an inflatable penile prosthesis, the method comprising:
detecting, by a controller of an electronic pump assembly, an external signal, the controller including a printed circuit board assembly, the printed circuit board assembly including a microprocessor or a state machine;
determining to activate an inflation cycle of the inflatable penile prosthesis based on the external signal; and
actuating at least one of a pump or an active valve to transfer fluid from a fluid reservoir to an inflatable member.

20. The method of claim 19, further comprising:
charging a battery of the electronic pump assembly based on wireless transmissions received via an antenna of the electronic pump assembly, the wireless transmissions including inductive, ultrasonic, near-field communication (NF), radio frequency (RF), or capacitive transmissions.
